(19) United States
(12) Patent Application Publication
(10) Pub. No.: US 2021/0252196 A1
(43) Pub. Date: Aug. 19, 2021

(54) TISSUE INTEGRATED DRUG DELIVERY SYSTEM

(71) Applicant: THE REGENTS OF THE UNIVERSITY OF CALIFORNIA, Oakland, CA (US)

(72) Inventors: Todd J. Thorson, Irvine, CA (US); Luciano Groisman, Irvine, CA (US); Ali Mohraz, Irvine, CA (US); Elliot L. Botvinick, Irvine, CA (US)

(21) Appl. No.: 16/972,972

(22) PCT Filed: Jan. 16, 2019
(86) PCT No.: PCT/US19/13825

§ 371 Date: Dec. 7, 2020

Related U.S. Application Data
(63) Continuation-in-part of application No. PCT/US18/36787, filed on Jun. 8, 2018.

(51) Int. Cl. A61L 31/14; A61M 5/158; A61M 25/00

(52) U.S. Cl. CPC A61L 31/14; A61M 5/158; A61M 25/0054; A61M 2025/0059; A61L 2420/04; A61L 2420/02; A61M 25/0009

(57) ABSTRACT

Infusion sets for subcutaneous drug delivery are described herein. The infusion set integrates a bijel-templated material (BTM) into a cannula such that a portion of the BTM protrudes from the cannula tip into the host tissue. The BTM is a porous, polymeric sponge having a co-continuous architecture with consistent curvature throughout non-constricting, interpenetrating channels, which is critical in mitigation of the deleterious host tissue response, vascularization, and flow redistribution in the implant.

### CROSS-REFERENCES TO RELATED APPLICATIONS

This application is continuation-in-part and claims benefit of PCT Application No. PCT/US18/36787, filed Jun. 8, 2018, the specification(s) of which is/are incorporated herein in their entirety by reference.

### BACKGROUND OF THE INVENTION

### i. Field of the Invention

The present invention relates to medical devices for infusion of drug solutions, for example, to insulin infusion sets that integrate a biodegradable bijel-templated material (BTM) having unique morphological characteristics into a cannula.

### ii. Description of Related Art Including Information Disclosed

Type 1 Diabetes (T1D) is an autoimmune disease affecting an estimated 1.25 million people in the United States, and roughly 1 million people manage their blood-glucose using continuous subcutaneous insulin infusion (CSII) pumps. These pumps are rapidly improving from patient-controlled insulin delivering machinery to bi-hormonal, fully automated closed loop algorithms constantly fluctuating insulin administration for complete and accurate blood-glucose regulation. Every few minutes, a decision is made by either the patient or the pump to delivery insulin, requiring large degrees of accuracy and reliability.

Insulin infusion sets (IIS) are devices that are used as the conduit to deliver insulin, or another drug such as glucagon or Pegfilgrastim®, from the reservoir of a CSII pump therapy system to the subcutaneous tissue. For most pump therapies, IISs are used to transfer the drug across the skin using either a steel hollow needle or a flexible polymeric cannula that is inserted using a steel needle or lancet, which is later removed leaving the cannula to remain to traverse the skin. The cannula is either connected directly into the insulin pump in tubeless systems or to a thin flexible tube routing back to a luer-lock or other proprietary connecter directly into the insulin reservoir within the insulin pump.

Commercial IISs on the market all have a similar mechanism where the insulin is delivered from the end of the cannula. After delivery, surrounding vasculature slowly absorbs the protein into the circulatory system and transports it throughout the rest of the body. Problems arise when this delivery chain is impeded, and insulin is unable to be picked up by the circulatory system. This can happen either with a kink of the cannula or a foreign body response (FBR) building fibrotic tissue to encapsulate the implant and preventing the drug from diffusing further. Due to the risk of infection and unresponsive insulin administration, commercial units are recommended to be changed every 2-3 days. This frequent change of sites and deposition of insulin into the subcutaneous tissue causes an increased amount of fat and scar tissue build up, and may result in a condition known as lipohypertrophy. These lumps under the skin are not only unsightly, but also painful and change timing and effectiveness of insulin. Issues such as these can lead to hyperglycemia where blood-glucose levels rise above safe levels leading to headaches, confusion, coma, or even death. Alternatively, if the insulin that has been trapped is then released and absorbed, an overdose of insulin may lead to hypoglycemia where blood-glucose levels drop below safe levels and side effects include seizures, loss of consciousness, and death.

While in the last several years there has been significant improvement in CSII sensing and pump technology for the management of diabetes, there has been little advancement for infusion sets. The need for new infusion set technology is regularly requested by patients and advocates who argue IIS improvements are long overdue. The present invention proposes a novel technology that can address the limitations of commercial cannulas.

### BRIEF SUMMARY OF THE INVENTION

It is an objective of the present invention to provide infusion systems and methods that allow for extended lifetimes and improved reliability, as specified in the independent claims. Embodiments of the invention are given in the dependent claims. Embodiments of the present invention can be freely combined with each other if they are not mutually exclusive.

In some aspects, the present invention features a novel infusion system for subcutaneous drug delivery. In a preferred embodiment, the infusion system integrates a unique material structure known as a bijel-templated material (BTM) into the interior of a cannula, where a portion of the BTM may protrude from the cannula tip into the host tissue. The BTM is a porous material that boasts non-constricting, interconnected similar sized channels throughout its volume. The infusion system has numerous advantages that include, but are not limited to, delaying or suppressing the body's own mechanism of fighting off implant materials, and preventing kinking and/or the effects of kinking.

Without wishing to limit the invention to any theory or mechanism, the protrusion is the site of beneficial tissue integration, deep vascularization, and flow redistribution. Biocompatibility and the unique channel structure of the BTM may reduce the foreign body response (FBR). The penetrating network of curved channels may provide a labyrinth-like network of connected paths for immune cells responding to normal wound inflammation. The consistent curvature within these channels and at the outer material surface has been shown to inhibit cells from forming a dense tissue layer at the host-material interface, thereby disrupting widespread encapsulation of the implant. In addition, the continuous, interconnected channels of the BTM provide multiple outlets that allow the mechanism of drug delivery to take alternative paths into the tissue, thereby preventing impeded flow. The interconnecting channel network also provides non-constricting paths for newly formed blood vessels to form a dense, mature vasculature within the BTM pores, thereby increasing the surface area for rapid absorption of insulin and other drugs.

Moreover, the filled portion of the cannula can provide resistance to kinking. By placing the BTM inside the cannula, this configuration may provide greater structural support to the cannula, thereby allowing flexibility without creating a kink and impeding flow. In other aspects, the BTM may be left behind in the tissue where it can degrade over time. None of the presently known prior references or work has the unique inventive technical feature of the present invention. Furthermore, the inventive technical features of the present invention contributed to a surprising result. Notably, the BTM implant outperformed a random porous scaffold, which is widely considered to be a breakthrough material, as assessed by the density of new blood vessels and the host inflammatory response. The degree of improvement was surprising and far beyond what was expected.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a non-limiting embodiment of a bijel-templated material (BTM)-loaded cannula of the present invention.

FIG. 1B shows a traditional infusion cannula.

FIG. 2 shows a non-limiting schematic of bijel formation. Particles are dispersed in the mixed fluid phase at room temperature and self-assemble on the fluid interface during spinodal decomposition. The bijel becomes kinetically arrested during bijel formation when particles completely occupy the fluid interface.

FIG. 3 shows a confocal microscopy image of a bijel.

FIG. 4 shows a non-limiting procedure for creating a BTM.

FIG. 5 shows an example of a short continuous path through an interconnected void in a BTM structure (left), and all possible interconnected paths throughout the polymer void (right).

FIGS. 6A-6C show non-limiting embodiments of manufacturing the BTM-loaded cannula.

FIG. 7 is a graph of domain size distribution in BTMs, compared to other commonly used porous materials.

FIG. 8A shows an insulin infusion set with a BTM-loaded cannula.

FIG. 8B shows a BTM-loaded cannula inserted into gelatin and dyed water delivered via insulin pump. Flow was redistributed throughout the BTM and delivered at least 1 mm downstream of the tube.

FIG. 9 shows a cannula loaded with the BTM porous polymer that resists kinking when bent at 90°. The cannula was bent 9 mm from the distal end of the cannula.

FIG. 10A is a schematic of a pressure testing setup of a BTM plug inserted inside a polyolefin tube equipped with pressure transducers at the inlet and outlet.

FIG. 10B is a graph of water flow rates through cannulas inserted with a BTM (square) or a random porous material (circle), as a function of the applied pressure drop.

FIG. 10C is a graph of pressure evolution during the alarm test.

FIGS. 11A-11B shows non-limiting examples of imparting biodegradable functionality to the BTM, including synthesis of materials susceptible to hydrolytic (FIG. 11A) or enzymatic (FIG. 11B) degradation.

FIG. 12 shows a non-limiting schematic of a cannula insertion procedure.

FIGS. 13A-13B show template schematics and scanning electron micrographs of BTM (FIG. 13A) and particle-templated material (PTM) (FIG. 13B) implants. The inset in FIG. 13B is zoom-in view showing larger pores in gray outlined in white having small interconnecting windows in black. Scale bar: 100 μm.

FIGS. 14A-14E show immunohistochemistry (IHC) and histological analysis of tissues to analyze FBR and vascularization potential of BTM and PTM implants. FIGS. 14A and 14B show confocal microscopy images of macrophage IHC for BTM and PTM implants, respectively. F4/80 macrophage surface marker is labeled in green, CD206 M2 polarization surface marker is labeled in red, and cell nuclei are labeled in blue (DAPI). An implant-tissue interface is shown in dashed white lines. FIGS. 14C and 14D show Masson's trichrome histology sections for BTM and PTM implants, respectively. Black arrows denote collagen deposition near tissue-implant boundary. Scale bar: 100 μm. FIG. 14E shows the percentage of CD206 labeled macrophages in BTM and PTM implants. Colored data represent three separate mice used in study. *p<0.05.

FIGS. 15A-15E show confocal microscopy images of α-SMA and CD31 labeled tissue sections. FIGS. 15A and 15B show vessel IHC demonstrating penetration in BTM and PTM implants, respectively. α-SMA in pericytes and myofibroblasts are labeled in green, CD31 in endothelial cells are labeled in red, and cell nuclei are labeled in blue (DAPI). Pericytes at vessel walls in the BTM indicate mature vasculature. The implant-tissue interface is shown in dashed white lines. An example of a constricted vessel in the PTM implant is denoted by a white arrow (FIG. 15B). FIGS. 15C and 15D show vessel area versus distance to tissue-implant boundary in BTM and PTM implants, respectively. Colored data represent three separate mice used in the study. FIG. 15E is a BTM implant image containing the largest vessel observed in the study, denoted by white star. Scale bar: 50 μm.

### DETAILED DESCRIPTION OF THE INVENTION

Following is a list of elements corresponding to a particular element referred to herein:
100 device
110 cannula
112 cannula lumen
114 cannula internal surface
116 cannula tip
118 cannula opening
120 implant
122 implant portion in cannula
124 protruding portion of implant
210 bijel
220 precursor
230 photoinitiator
240 prepolymer
250 BTM
330 BTM outlet/opening
335 BTM surface
350 BTM continuous path
360 BTM volume

Bijel-Templated Materials (BTM)

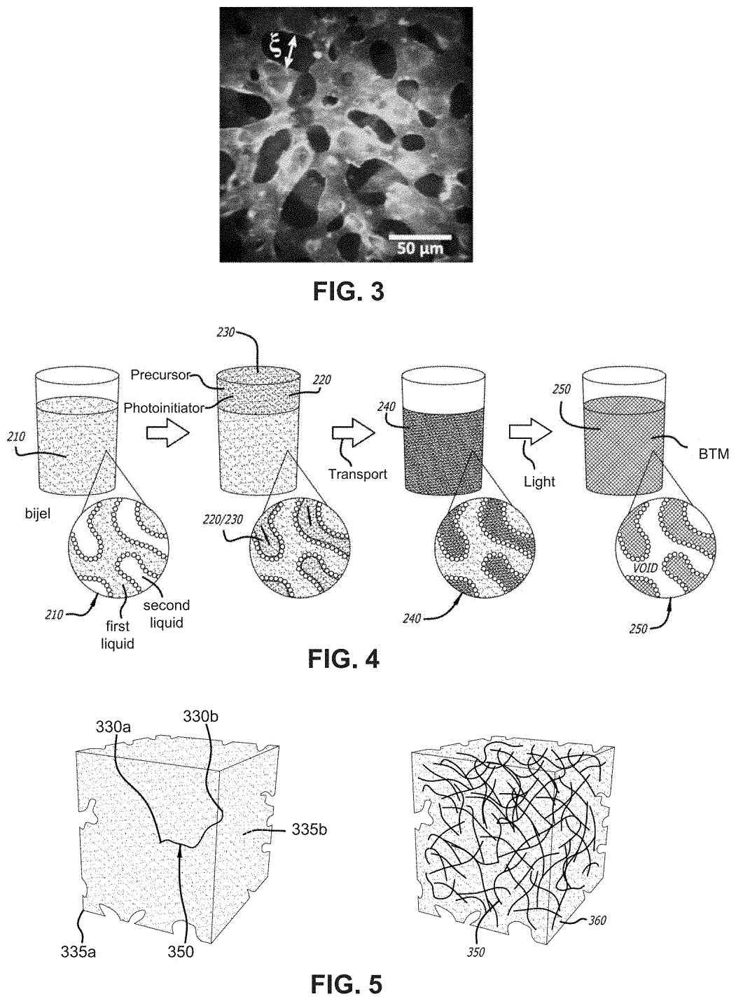

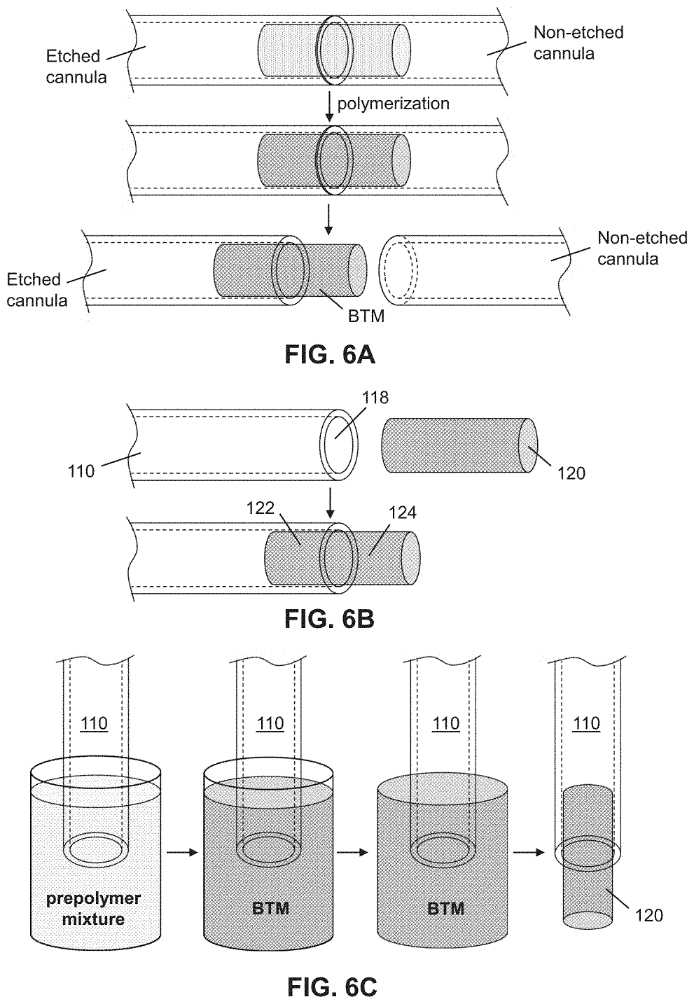

Referring to FIG. 2, the unique morphology described in this invention utilizes a class of soft materials known as bicontinuous interfacially jammed emulsion gels, termed herein as “bijels”. The formation of these soft materials occurs through arrested phase separation of a binary liquid mixture undergoing spinodal decomposition in the presence of neutrally wetting colloidal particles. In some embodiments, the bijel mixture undergoes a temperature change (e.g., increased application of heat for a selected period of time) that brings the bijel mixture to or past a critical temperature and induces spinodal decomposition phase separation. During phase separation, the particles adsorb to the fluid-fluid interface (early stage), and the system becomes jammed as the interfacial area is sufficiently reduced to just accommodate particles (late stage). The resulting soft material is comprised of two bicontinuous and interpenetrating fluid domains. The size of the subsequent self-similar domains, or micro-channels, can be tuned over a biologically relevant range (5 μm to 150 μm) solely through the volume fraction of particles in the system. Through varying this characteristic, micro-channel domain size, the internal local curvature, which is implicated in tissue response, is consequently varied. Without wishing to limit the present invention, the bijels can form a mechanically stable, tubular arrangement of two fluid phases with the following unique morphological characteristics: 1) the tubular arrangement is bicontinuous, providing uninhibited paths for molecular and cellular transport through each phase, throughout its volume; 2) the characteristic domain size (the diameter of the tubular domains, ξ in FIG. 3) is nearly uniform throughout the mixture, and importantly, can readily be tuned over a relatively wide range of 5 μm<ξ<150 μm; and 3) the interface separating the two fluids is a continuous 3D surface with a predominance of negative Gaussian curvatures (saddle points), devoid of corners, kinks, or edges.

Referring now to FIG. 4, the kinetically stable bijels (210) can be transformed into a polymer/void construct by exploiting the incompatible chemistries of the two fluid domains, through selectively polymerizing one phase. For example, a monomer that is selectively soluble in one of the bijel fluid phases is mixed with a photoinitiator and placed on top of the bijel and allowed to diffuse into that particular phase, and the BTMs may then be formed through polymerization. Briefly, a precursor (220) (e.g., monomer or material precursor) which may also be mixed with a substance (230) (e.g., photoinitiator that creates a reactive liquid phase) is placed on top of the bijel (210) and allowed to transport (e.g., diffuse) preferentially into one of the liquid domains, as dictated by the precursor solubility within each phase. The BTM (250) may be formed by photopolymerization of the precursor-containing liquid phase (240), in response to exposure of the photoinitiator (230) within the bijel to the appropriate wavelength and dosage of light. After photopolymerization, if necessary, any excess polymer not exhibiting the bijel-templated morphology may be removed and unreacted materials may be removed through washing with isopropyl alcohol or other suitable solutions. Alternatively, the BTM (250) may be formed by another type of polymerization in lieu of the application of light (e.g., thermally activated, chemically activated, time-based activation, or irradiation) based on the type of precursor (220) added to the bijel and/or type of substance (230) added.

In a non-limiting embodiment of bijel formation, a variety of binary fluid systems that undergo spinodal decomposition and particles can be used. For instance, a solution of water/2,6-lutidine and silica particles (D˜500 nm) may be used for bijel formation. However, the binary fluid system is not limited to this solution, and it is understood that a wide variety of other materials can be used. The interconnectivity of the bijel is imparted onto the polymer using the disclosed polymer processing method. In one embodiment, the bijel can be transformed into a bicontinuous hydrogel through selectively polymerizing the lutidine rich phase containing a hydrophobic monomer mixed with an oil soluble photoinitiator. As an example, polyethylene glycol diacrylate, Mn:258 (PEGDA 258) and Darocur 1173 may undergo photopolymerization for approximately 45 seconds. In another embodiment, the silica particles can then be removed through a hydrofluoric acid etch, leaving only the cross-linked polymer. In an alternative embodiment, the BTMs can be fabricated by adding the material precursor directly to the water/2,6-lutidine/silica nanoparticle mixture before bijel formation.

The resultant polymer morphology can be analyzed using various imaging modalities such as digital microscopy, scanning electron microscopy (SEM), and computed tomography (CT). As shown in FIG. 5, high resolution three-dimensional renderings obtained via CT allows for complete analysis of the unique morphology imparted onto the polymer by the bijel precursor. For example, FIG. 5 shows a 1 mm³ rendered volume of a bijel-templated PEG hydrogel. The shortest continuous path (350) between a first opening (330a) of the void domain at one surface (335a) of the BTM and a second opening (330b) of the void domain located at another (e.g. opposite) surface (335b) of the BTM can be computed from CT scans. Further, the CT scan can be used to calculate all possible continuous paths (350) throughout the entire polymer volume (360). These results exhibit the unique connectivity of the void domains, and similar results are found when computing shortest continuous paths within the polymer domain. The connectivity of the material domains is important to this invention because cells, fluids, nutrients, etc. do not encounter dead ends throughout the polymer. Without wishing to limit the present invention, the BTM has the advantages of uniform pore size distribution; notably the absence of constricting windows, which aids in flow redistribution and allows for the formation of larger vessels at the scale of the BTM pore size. SEM micrographs of BTM and particle templated material (PTM) implants pictured in FIGS. 13A-13B exemplify these contrasting pore features, where the dark regions are the pores. Additional examples and details of the bijel and BTMs are described in PCT Application No. PCT/US18/36787, the specification(s) of which is/are incorporated herein in their entirety by reference.

BTM Infusion Sets

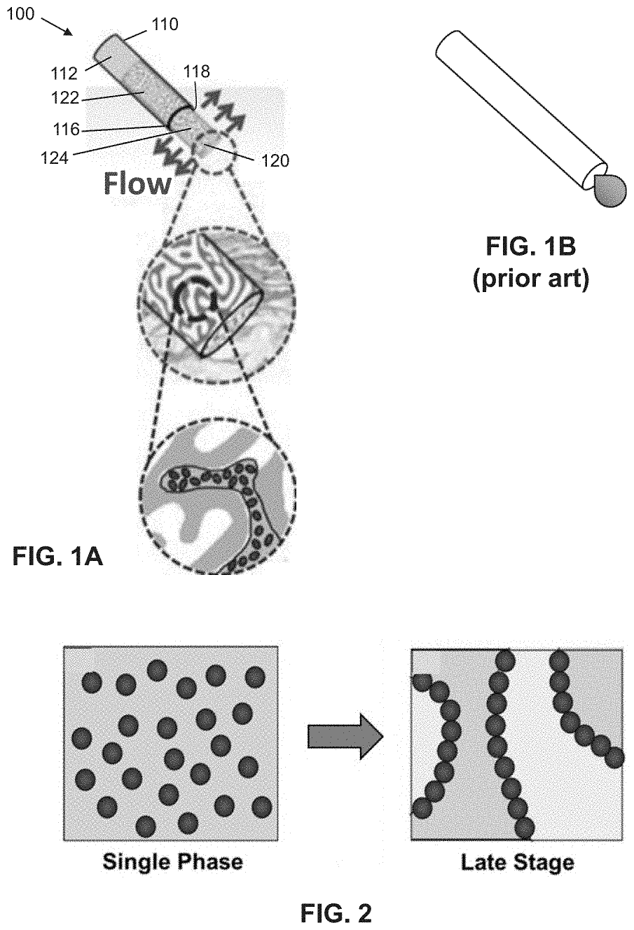

Referring now to FIG. 1A, the present invention features a biomedical device (100) comprising a cannula (110) loaded with a porous material (120). Preferably, a portion (122) of said porous material is disposed inside the cannula and a remaining portion (124) thereof is protruding from a tip (116) of the cannula. The portion (122) of the porous material disposed inside the cannula may be bonded or mechanically affixed to an internal surface (114) of the cannula. In some embodiments, the porous material (120) may be comprised of a bijel-templated material (BTM) that had continuous, interconnected channels (350) with multiple perfusion outlets (330).

According to another embodiment, an infusion device of the invention may comprise a cannula (110) having a lumen (112) and an opening (118) disposed on one end of the cannula, and fluidly connected to said lumen (112), and a material implant (120) having a portion (122) thereof disposed within the lumen (112) and a remaining portion (124) thereof protruding from the opening (118) of the cannula. The material implant (120) may be constructed from a porous BTM having continuous, interconnected channels (350) with multiple perfusion outlets (330).

In other embodiments, the present invention features an infusion system comprising a cannula (110) having a tubular body with a proximal end, a distal end, and a lumen (112) extending between said ends, a porous material implant (120) having a portion (122) thereof disposed within the lumen (112) and a remaining portion (124) thereof protruding from an opening (118) of the distal end, and a pump fluidly coupled to the proximal end of the cannula. The porous material implant (120) may comprise a BTM having continuous, interconnected channels (350) with multiple perfusion outlets (330).

Without wishing to limit the present invention, the implant (120) of the present invention can prevent kinking of the cannula. For example, the portion (122) of the implant disposed within the lumen (112) may be bonded or mechanically affixed to an internal surface (114) of the cannula. This portion (122) of the material implant may prevent kinking.

In accordance with the implants described herein, at least a portion (122) of the implant disposed within the lumen (112) may be cylindrical in shape. Preferably, this portion is shaped and sized so as to fit within the lumen. For instance, a diameter of the implant, either the portion (122) disposed within the lumen (112) or the portion protruding (124) from the lumen (112), may be about equal to the diameter of the lumen. Alternatively, the diameter of the implant may larger or smaller than the diameter of the lumen. Non-limiting examples of lumen diameters range from about 0.2 mm to about 5 mm. In other embodiments, the protruding portion of the lumen may be cylindrical in shape. However, other shapes may also be suitable for the infusion device, such as a spherical or tapered shape.

In some embodiments, the implant may be about 0.5 cm to about 1 cm in length. In other embodiments, the implant may be about 0.8 cm to about 2 cm in length. In some other embodiments, the implant may be about 2 cm to about 4 cm in length. It is to be understood that these lengths are non-limiting examples only, and that any suitable length of the implant may be used in accordance with the present invention. In one embodiment, the implant may be disposed within the cannula such that about half of the implant is inside the cannula and the remaining half is protruding from the cannula. For example, about 0.5 cm of a 1 cm long implant may be disposed in the cannula. In another embodiment, about 25% to 75% of the implant may be disposed within the cannula and the remaining portion is protruding from the cannula. To illustrate, about 0.5-1 cm of a 2 cm long implant may be disposed within the cannula and the remaining portion is protruding from the cannula.

It is critical to operation that a BTM has the microstructural properties unique to its bijel template, throughout its entire volume. Such continuity is required in order to achieve the benefits of the present invention, including preventing pore blockage and detachment from cannula, accurate insulin delivery, and operation within safe pressure ranges of commercial insulin pumps.

As shown in FIG. 1B, commercial infusion sets only dispense fluid from the end of the cannula, similar to water flowing out of a hose. This pressurized volume of delivered fluid collapses the local microcirculation, impeding drug absorption into the body. Buildup of fibrotic tissue from the FBR to the cannula can encapsulate the cannula tip, thus further impeding drug absorption. Together these two effects can detrimentally reduce the drug's effectiveness. Furthermore, the typical wear time of a commercial infusion set is about 2-3 days causing tissue trauma and remodeling with every insertion.

The unique microstructure of the present invention allows for an extraordinarily large quantity of paths for the fluid to take as shown in FIG. 1A and FIG. 5. In addition to their quantity, the geometry of the channels in the present invention offers advantages for fluid transport. Particularly, the present invention uniquely offers a uniform micro-channel geometry that also results in a network of non-constricting, fully penetrating curved channels with roughly uniform diameter. This results in flow redistribution such that the drug solution is delivered along the entire length of the device protrusion. For example, as shown in FIG. 8B, the redistribution of flow may be orthogonal to the axis of the cannula which allows for multiple fluid paths and decreased local pressure.

Moreover, the present invention can promote vascularization into the delivery device, allowing drugs to come in contact with a larger total surface area of vessels, and thus increasing effectiveness of the drug delivery. Furthermore, if encapsulation does begin, vessels within the microstructure may still effectively continue the drug therapy. Without wishing to be bound to a particular theory or mechanism, the infusion set of the present invention can elicit a reduced FBR as indicated by lack of fibrotic tissue at the host-implant interface. By mitigating the FBR, usage time could be increased resulting in less trauma and scar tissue.

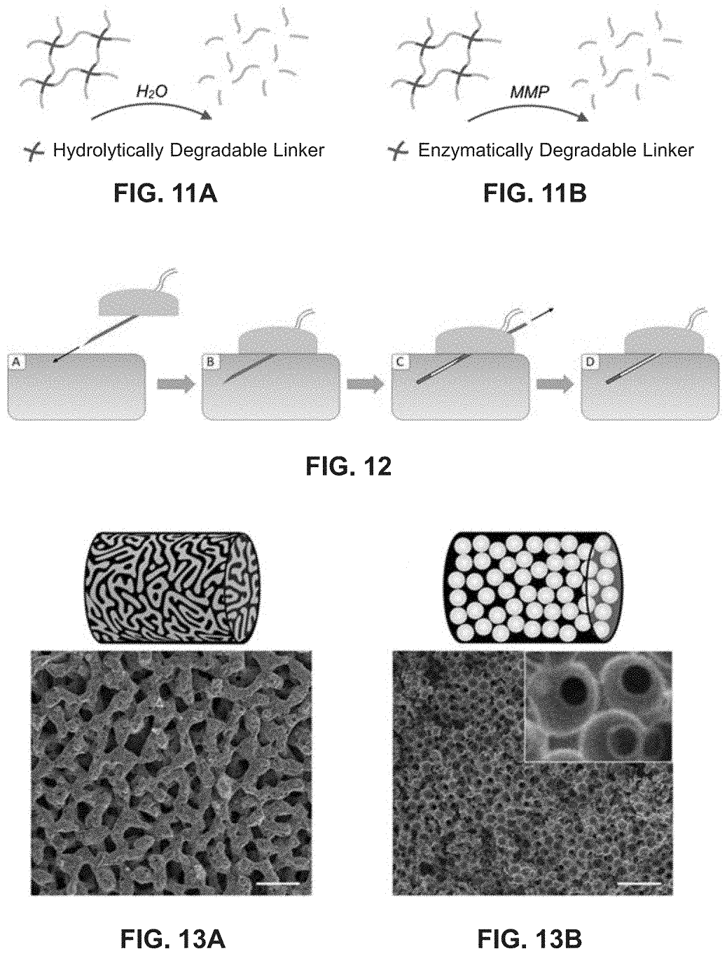

In some embodiments, the BTM may be prepared from a variety of hydrogel or polymer precursors. Without wishing to limit the present invention, the BTM precursors can preferably meet the following characteristics: 1) biodegradability of the polymerized product, 2) selective solubility in one of the two bijel fluid phases, 3) biocompatibility and low cytotoxicity, 4) hydrophobicity of the polymerized product, and 5) ability to bond to existing cannula materials. Of particular interest is the biodegradability of the BTM, which may be left behind upon removal of the cannula due to tissue infiltration. Therefore, the precursor materials are selected such that BTM can sufficiently maintain its structural integrity over 14 days or longer, and the BTM fragments left behind after removal can degrade safely over a time span of several weeks. Strategies to impart biodegradable functionality to the BTM may include hydrolytically degradable co-polymer linkers (e.g. poly (lactic-co-glycolic acid), FIG. 11A) or enzymatically degradable linkers (e.g. matrix metalloproteinase (MMP) sensitive peptides, FIG. 11B). Such strategies may enable tuning of the degradation kinetics through the concentration of biodegradable sites within the BTM.

According to some embodiments, the BTM can be formed from a kinetically stable bijel by exploiting the incompatible chemistries of the two liquid domains and selectively replacing (partially or entirely) at least one of the liquid domains with an alternative material. For example, a liquid not having optimal characteristics for the formation of a bijel, may be integrated into the bijel following particle jamming and stabilization. In another embodiment, a monomer or material precursor mixed with a photoinitiator may be placed on top of the bijel and allowed to transport preferentially into one of the liquid domains, as dictated by the precursor solubility within each phase. For example, the precursor may comprise PEGDA and a photoinitiator.

Without intending to limit the present invention, a wide variety of precursors may be used to create the BTM so long as the precursor is solely solubilized by one of the liquids of the bijel, each liquid of the bijel can either be one of the liquids used to form the bijel, or a liquid subsequently replacing (either partially or in part) one of the liquids used to form the bijel. The precursors may contain a polymerizable component. BTMs may comprise biocompatible materials including, but are not limited or restricted to, polyethylene glycol (PEG), poly(hydroxyethylmethacrylate) (PHEMA), polycaprolactone (PCL), and polylactide (PLA). Furthermore, a BTM (e.g. one made of PEG) may be used as a skeletal structure available for the casting of additional materials. These materials may include, but are not limited to, zwitterionic hydrogels comprised of poly(carboxybetaine methacrylate (PCBMA), PDMS, poly(N-vinylpyrrolidone) (PVPON), poly(N-isopropylacrylamide) (PNIPAM), polytetrafluoroethylene (PTFE), or copolymers containing biodegradable or photodegradable blocks.

Fabrication and Use of BTM Infusion Sets

Various methods may be implemented to fabricate the biomedical device of the present invention. These methods can depend on the BTM and/or the cannula material.

In some embodiments, the method may comprise inserting a porous material (120) into a cannula (110) such that a portion (122) of said porous material (e.g. BTM) is disposed inside the cannula (110) and a remaining portion (124) thereof is protruding from a tip (116) of the cannula. Preferably, the porous material (120) is formed to a shape that can fit inside the cannula (110). In one embodiment, the portion (122) of the porous material disposed inside the cannula may be mechanically affixed to an internal surface (114) of the cannula. As an example, the cannula (110) may be shrunk so that the cannula constricts around the portion (122) of the porous material disposed inside the cannula. In another embodiment, the porous material (120) may be bound to an internal surface (114) of the cannula (110). For instance, the porous material (120) may be covalently bound to the internal surface (114) of the cannula (110). An adhesive may be used to bind the porous material (120) to the internal surface (114) of the cannula (110).

According to other embodiments, the method of fabricating the infusion device may comprise placing a prepolymer mixture in a cannula (110), and polymerizing said prepolymer mixture to form a porous material (120) such that a portion (122) of said porous material is bound to an internal surface (114) of the cannula (110) and a remaining portion (124) thereof is protruding from a tip (116) of the cannula. The method may further include shaping the protruding portion (124) of the porous material to a desired shape and size. In some embodiments, the prepolymer mixture forms the BTM porous material (120) having continuous, interconnected channels (330) with multiple perfusion outlets (350). The prepolymer mixture may comprise a bijel-templated mixture that includes a plurality of particles, a first liquid, and a different second liquid that is partially miscible with the first liquid. The second liquid may contain monomer precursors. The BTM is produced by polymerizing the bijel-templated mixture such that the monomer-containing second liquid is polymerized. In some embodiments, the plurality of particles and the first liquid can be removed after polymerization.

The unique processing techniques of this invention allows for biomaterial preparation with minimal steps and energy requirements, while offering a scalable route to semi-continuous device manufacturing. The invention is fabricated with inexpensive solvents and particles heated quickly to a modest temperature. The processing steps allow for material selection from a wide variety of polymerizable material precursors. Through the afforded variability of material chemistries, functionalization with any number of important bio-active signals is made possible.

In some embodiments, the BTM may be formed in situ within and protruding from the infusion cannula. In one embodiment, a requirement for the BTM precursor is the ability to covalently bond to typical commercial IIS materials. Possible routes include, but are not limited to, radical polymerization to surface etched polytetrafluoroethylene (PTFE) or Michael-type addition to amine-containing materials. Michael-type addition reactions are known to one of ordinary skill in the art.

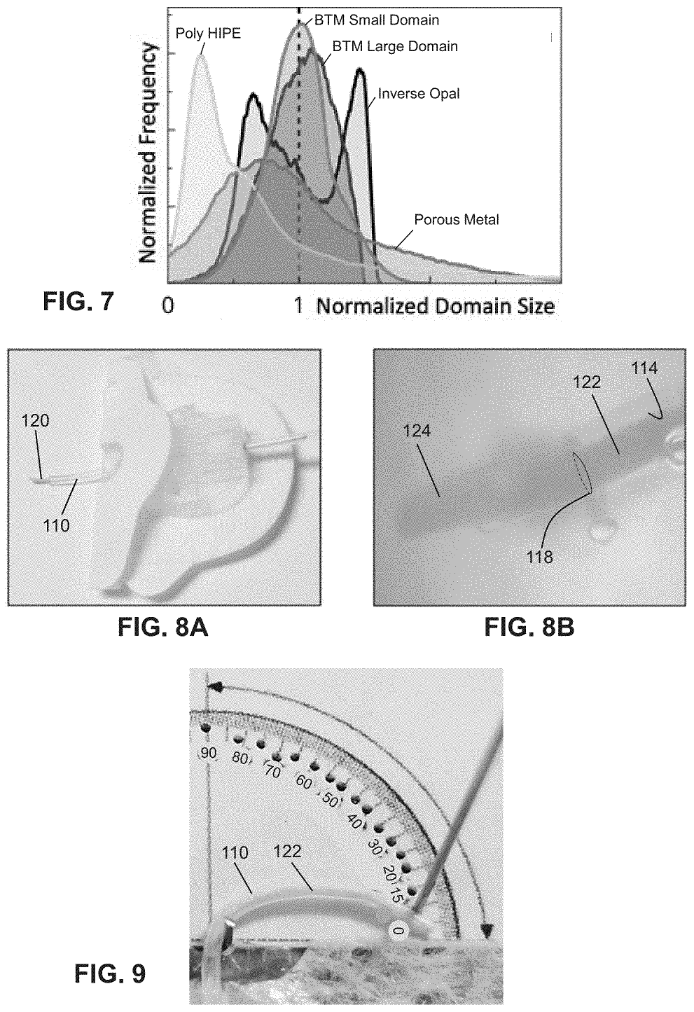

In a non-limiting example, as shown in FIG. 7A, an inner surface of the PTFE cannula may be treated with sodium naphthalene to allow for covalent bonding to the BTM. Fluorine atoms are removed during the etching process leaving chemical groups available for bonding through radical polymerization of acrylate-containing monomers or material precursors. The PTFE cannula with an etched inner lumen may be arranged with the tip co-registered to the tip of a non-etched PTFE cannula, and the BTM is formed using polymerization within the inner lumens. The BTM in this case is now covalently bonded to the etched PTFE cannula only, and a protruding portion of the BTM is formed by pulling away the non-etched cannula. In other embodiments, one of the two cannulas may have an amine-containing inner luminal surface and the BTM is covalently bonded to the cannula using a Michael-type addition reaction.

In some embodiments, the cannula may be constructed from materials that include, but are not limited to, PTFE (after removing a fraction of fluorine through common etching techniques) and surface activated variants of polyvinyl chloride (PVC), polyurethane (PU), polydimethylsiloxane (PDMS), polyether ether ketone (PEEK), or polyethylene. In other embodiments, photopolymerization may be utilized to form the BTM and bind the BTM the cannula. An alternative type of polymerization (e.g., thermal, chemical, time-based, or another type of irradiation) may be used with a suitable precursor or initiator being added to the system.

In another embodiment, as shown in FIG. 7B, a BTM structure may be manufactured and then formed into a cylindrical shape having a desired diameter or cut to desired dimensions. The shaped BTM structure can then be placed in the lumen of a flexible cannula such that a portion of BTM structure is protruding from the tip. The BTM portion that is placed in the cannula lumen is then covalently bonded to the cannula using a number of different chemistries. Possible routes include, but are not limited to a first embodiment, in which the BTM is covalently bonded to an inner surface-etched PTFE cannula by radical polymerization and a second embodiment in which a cannula is selected having an amine-containing inner luminal surface and the BTM is covalently bonded to the cannula using a Michael-type addition reaction.

In yet another embodiment, as shown in FIG. 7C, a portion of the cannula near the tip may be placed in a BTM prepolymer mixture. The mixture is then polymerized to form a solid BTM structure. The BTM structure may then be formed into desired dimensions such that a portion of BTM structure is protruding from the tip. In some embodiments, the inner surface of the cannula may be etched. In other embodiments, the cannula may have an amine-containing inner luminal surface and the BTM is covalently bonded to the cannula using a Michael-type addition reaction.

According to other embodiments, the infusion system of the present invention may be used to infuse a fluid into a subject. A non-limiting example of an infusion method may comprise inserting at least the portion of the porous material implant protruding from the distal end of the cannula into a tissue of the subject, and pumping the fluid into the cannula via the pump such that the fluid flows through the lumen and exits through the protruding portion of the porous material implant, thereby infusing the subject with the fluid. The insertion of at least the protruding portion into the subject's tissue includes insertion of the protruding portion or insertion of the protruding portion and at least part of the cannula with the porous material disposed therein. For instance, the protruding portion of the implant and about 0.5-2 cm of the cannula may be inserted into the tissue. The pumped fluid can exit through the protruding portion (124) of the implant or the entire portion of the implant that is in the tissue and, at least in part, the implant portion that is in the cannula that is itself in the tissue. In some embodiments, the BTM infusion sets may be implanted subcutaneously. The implanted parts of the BTM infusion set may be oriented laterally or perpendicularly with respect to the skin surface, or set at an angle ranging from about 25°-45°. While the BTM infusion set may be used for insulin infusion, it is to be understood that the present invention is not limited to insulin infusion and may be used for delivery of other solutions.

In one embodiment, as shown in FIG. 12, the cannula of the BTM infusion set may be inserted using a lancet insertion system. Briefly, the cannula is housed in a boat-shaped lancet which is integrated into an infusion set complete with tubing terminating in a luer lock plastic connector to be compatible with commercial insulin infusion pumps. The lancet is fabricated with a razor-sharp leading edge to cut through tissue as it is inserted into the skin. Next, the lancet is removed from the skin, leaving the cannula behind and within the tissue. The aforementioned is one example of implanting the cannula, and it is to be understood that other methods may be applied as known to one of ordinary skill in the art.

### Examples of the Present Invention

The following describes non-limiting examples of the present invention. It is to be understood that said examples are not intended to limit the present invention in any way. Equivalents or substitutes are within the scope of the present invention.

Flow Redistribution

To demonstrate flow redistribution, a BTM was placed in-line with an infusion set and held in place with shrink-tubing. Referring to FIG. 8A, a cannula was assembled by shrinking a polyolefin tubing around a polyethylene glycol (PEG) BTM. The cannula was inserted in gelatin, and dyed fluid was then pumped through the BTM-loaded cannula as shown in FIG. 8B. This experiment shows that the BTM pore structure permits flow at a rate of a standard Medtronic insulin pump. The dyed fluid was redistributed orthogonal to the axis of the BTM-loaded cannula throughout the BTM and uniformly at least 1 mm downstream of the tubing.

Kink testing was also performed with a porous polymer in the diameter of the interior of a cannula, which was shown to result in stability when the infusion set is bent. Kinking experiments were performed and designed around known modes of failure of commercial IIS. The tip of a cannula was bent 9 mm from the tip. In FIG. 9, when the cannula loaded with the porous BTM polymer was bent at 90°, it was able to maintain a supportive arch thereby preventing kinking.

Pressure Testing

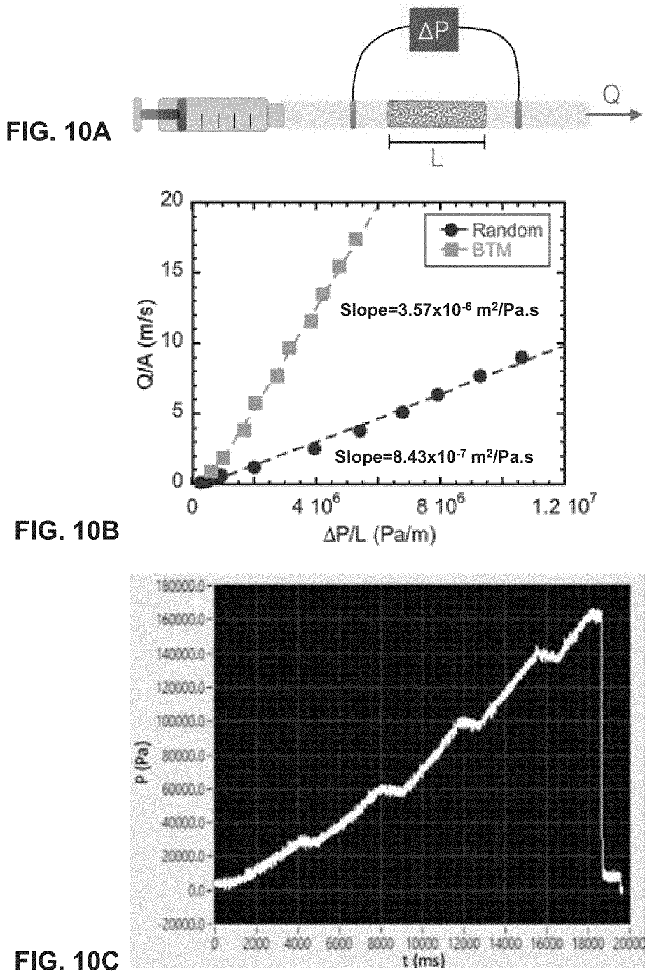

Insertion of a porous material inside a cannula is expected to result in changes to the flow kinematics and the pressure drop required to deliver a given volumetric flow rate of insulin through the cannula. However, the open and uniform pore morphology of BTMs may result in large hydraulic permeability and insignificant changes to the operating pressures required for insulin delivery at physiologically relevant flow rates. A pressure test was performed as follows: First, the permeability of a BTM made of polyethylene glycol was measured and compared to a random porous sponge of the same chemistry and length, and similar porosity (ϕ˜0.5 in both materials) with pores approximately 30-35 micrometers in diameter. Each plug was inserted inside a polyolefin tube equipped with pressure transducers at the inlet and outlet as shown in FIG. 10A. Water at various flow rates was pumped through the cannula, and the pressure drop required to sustain each flow rate was measured.

FIG. 10B shows a plot of the flow rate normalized by the tube inner cross-sectional area, versus the pressure drop per unit length of the tube. Assuming the data represents laminar flow of liquids through porous media, Darcy's law should hold:

$$Q = - \frac{kA Δ P}{μ L} ,$$

where Q is the liquid volumetric flow rate, A is the tube inner cross-sectional area, ΔP is the pressure drop, L is the tube length (8.5 mm in the experiment), k is the permeability, and μ is the liquid viscosity (8.9×10⁻³ kg/ms in the experiment). Therefore, the slope of each line in FIG. 10B provides a direct measure of k/μ, from which the material's permeability can be calculated. From the data in FIG. 10B, the following are obtained: k_{BTM}=3.16×10⁻⁹ m², k_{random}=7.50×10⁻¹⁰ m². Immediately, it is apparent that a bijel-derived porous material exhibits a higher hydraulic permeability than a random porous structure with comparable porosity.

Next, a test was performed to assess whether the anticipated pressure drop for insulin delivery through a BTM-inserted cannula falls within safe operating limits of current infusion pumps, and their associated commercial ISS. The alarm pressure of a Medtronic Silhouette infusion set was determined by step-wise addition of insulin (1 unit at a time) into a sealed tube that was initially filled with water, while monitoring the pressure, until the alarm was triggered. FIG. 10C shows the resulting step-wise pressure rise and the breakpoint (the test was repeated to ensure reproducibility). From FIG. 10C, the alarm pressure was determined to be Pₐₗₐᵣₘ=160 kPa. Finally, to calculate the anticipated pressure for delivery of insulin through a BTM-inserted cannula, Darcy's law was applied using the measured permeability and proposed length of the BTM (k_{BTM}=3.16×10⁻⁹ m², L=1.7 cm), the known cross-sectional area of a cannula (A=7.07×10⁻² mm²), and the maximum flow rate of the Medtronic Silhouette infusion set during bolus delivery (Qₘₐₓ=3.33×10⁻⁷ m³/s), which gives: ΔP=22.5 kPa (note this value is calculated with the maximum possible flow rate, giving an upper limit to the pressure drop). Therefore, the maximum anticipated pressure required for the delivery of insulin at physiologically relevant rates is 7× smaller than the alarm pressure in a current infusion set.

Reduced FBR

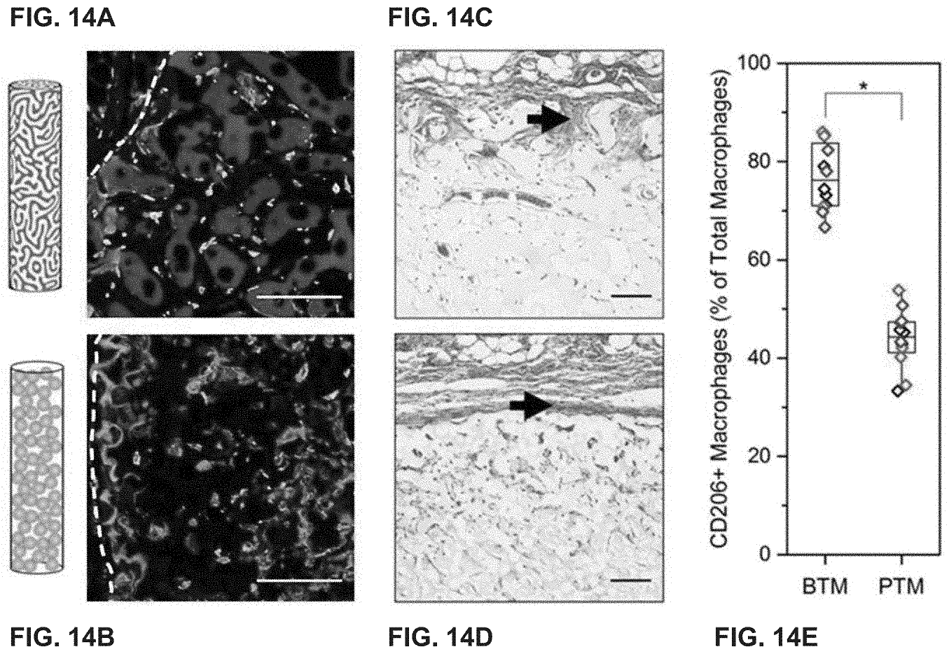

Four-week animal studies were performed in the subcutaneous space of athymic nude mice to analyze FBR and vascularization potential of BTM and PTM implants. Referring to FIGS. 14A-14E, immunohistochemistry (IHC) and histological analysis of tissues showed a reduction in the FBR in the BTM implants. Specifically, macrophage infiltration (F4/80+) was scattered throughout the pore network in the BTM (FIG. 14A) with many cells adhered to the implant material leaving much of the pore volume unoccupied. In contrast, macrophages were more spread and occupied a larger portion of the pore network within the PTM implants (FIG. 14B). The CD206 macrophage M2 polarization marker was observed in approximately 77% compared to just 44% of the F4/80+ cells counted in the BTM and PTM implants, respectively (FIG. 14E). Fibrotic collagen deposition at the implant-tissue interface was less pronounced and oriented in the BTM (FIG. 14C) than in the PTM implants (FIG. 14D). Additionally, diffuse collagen was deposited in a wound-healing fashion within the BTMs up to 500 μm from the interface.

Deep Vascularization

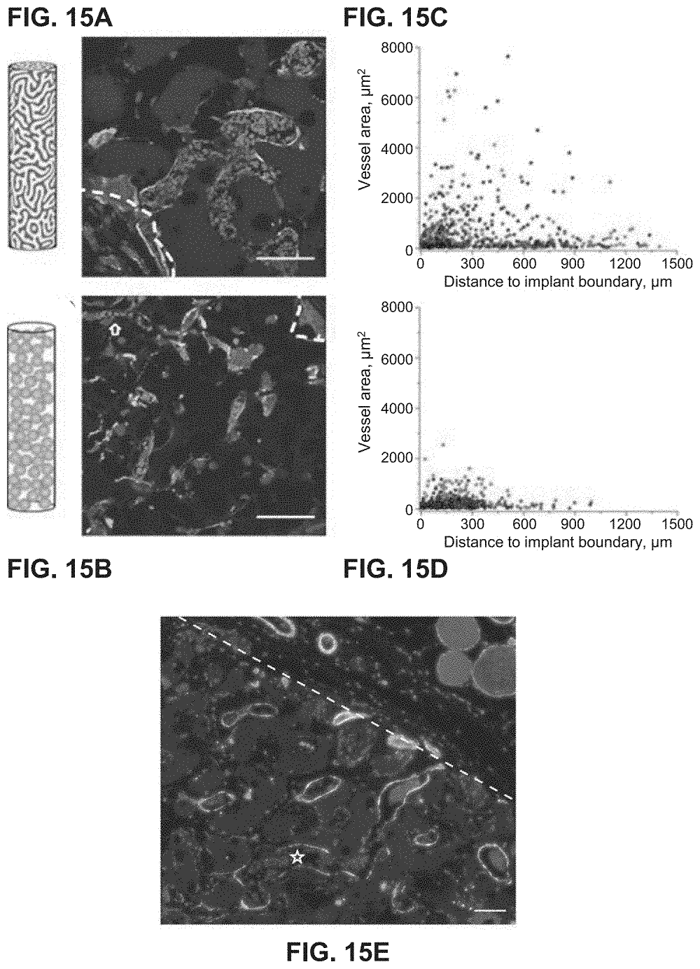

Confocal microscopy images of αSMA and CD31 labeled tissue sections are shown in FIGS. 15A-15E. Vessels within the pore network of the BTM implant (FIG. 15A, 15E) were both αSMA+ and CD31+, indicating mature vessels bound by pericytes. These vessels often completely occupied the pore network in the BTM. Conversely for PTM implants, thin vessels were often limited in diameter by the pore-to-pore windows (FIG. 15B) such that red blood cells could only pass in single file. Analysis of vessel area with respect to implant boundary showed far greater instances of large, deep vessels in the BTM (FIG. 15C) versus the PTM implants (FIG. 15D). The largest vessel observed extended from the boundary into the BTM implant ˜350 μm, growing to ˜22,000 μm² (FIG. 14E).

The stark difference in both FBR reduction and deep vascularization may be attributed to differences in the pore network microstructure present in each implant type. Specifically, the non-constricting nature of the BTM implant allowed blood vessels to not only form and occupy the entire micro-channel diameter, snaking along the curved interface, but also reside deep within the implant. In contrast, the constricting windows connecting the pores of the PTM implants force vessels to narrow, often leaving much of the pore volume vacant. As mentioned earlier, bijels self-assemble during spinodal decomposition, a phase separation process marked by dynamically self-similar, bicontinuous, fully percolating fluid domains. The resulting energetically preferred minimum surface area interface displays negative Gaussian, zero mean (hyperbolic or saddle) curvature. These attributes are transferred to the templated PEG implants resulting in pore networks that do not constrict, do not have any dead ends or non-utilized volume, and a surface displaying hyperbolic curvature.

The microstructure at the implant-tissue interface may also be responsible for disruption of dense collagen encapsulation as cells may not be able to span the alternating PEG-void structure. The microsphere templating process used to synthesize PTM implants relies on varying degrees of sphere fusion, which imparts interconnected pore windows upon template removal. The constrictions may also play a role in the nature of the tissue infiltration as pores were often packed with F4/80+ cells. Cell migration is impeded by the constricting nature of the pore windows, which may also influence macrophage polarization. In contrast, cells are allowed in infiltrate the labyrinth-like pore network of bijel-templated implants unobstructed by any constrictions. Allowing macrophages and other native cells to infiltrate without obstruction may lead to delay in the time to final fibrotic encapsulation, thereby extending the lifetime an implantable device. Additionally, the presence of large, mature vessels, even in the event of delayed collagen deposition at implant-tissue interface, could provide longer-term interaction with implantable tissues or infusion of a therapeutic.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited in the below claims are solely for ease of examination of this patent application, and are exemplary, and are not intended in any way to limit the scope of the claims to the particular features having the corresponding reference numbers in the drawings.

1.-30. (canceled)

31. An infusion device comprising:
a. a cannula having a lumen and an opening disposed on one end of the cannula, wherein the opening is fluidly connected to said lumen; and
b. a bijel-templated material having a first portion disposed within the lumen and a remaining portion protruding from the opening of the cannula.

32. The infusion device of claim 31, wherein the bijel-templated material comprises continuous, interconnected channels with multiple perfusion outlets.

33. The infusion device of claim 31, wherein the first portion of the bijel-templated material disposed within the lumen is bonded, at least in part, to an internal surface of the cannula.

34. The infusion device of claim 33, wherein the first portion of the bijel-templated material is bonded to the internal surface of the cannula that either has native functional groups or is activated by chemical means to form covalent bonds with complementary chemistries of the BTM.

35. The infusion device of claim 33, wherein the first portion of the bijel-templated material is bonded to the internal surface of the cannula by mechanically affixing the first portion of the bijel-templated material, at least in part, to an internal surface of the cannula.

36. The infusion device of claim 31, wherein the first portion of the bijel-templated material within the lumen prevents kinking of the cannula.

37. The infusion device of claim 31, wherein the cannula includes a proximal end, a distal end including the opening and the remaining portion of the bijel-templated material, and the lumen extending between the proximal end and the distal end.

38. The infusion device of claim 31 being deployed as part of an infusion system including a pump fluidly coupled to the proximal end of the cannula.

39. A method of fabricating an infusion device comprising:
a. placing a bijel-templated material into and protruding from or forming the bijel-templated material within and protruding from a cannula; and
b. binding the bijel-templated material to an internal surface of the cannula;
wherein a first portion of the bijel-templated material is disposed inside the cannula and a remaining portion of the bijel-templated material is protruding from an opening of the cannula.

40. The method of claim 39, wherein the binding of the bijel-templated material is conducted by at least covalently binding the bijel-templated material to the internal surface of the cannula, or by at least binding the bijel-templated material to the internal surface of the cannula by an adhesive.

41. The method of claim 39, wherein the binding of the first portion of the bijel-templated material is conducted by at least mechanically affixing the first portion of the bijel-templated material to at least the internal surface of the cannula.

42. The method of claim 39, wherein the placing of the bijel-templated material into the cannula is conducted by shrinking the cannula to constrict the first portion of the bijel-templated material.

43. The method of claim 39, wherein the forming of the bijel-templated material comprises placing a prepolymer mixture in the cannula and polymerizing the prepolymer mixture to form the bijel-templated material with the first portion of the bijel-templated material being bound to an internal surface of the cannula and the remaining portion of the bijel-templated material protruding from the opening of the cannula.

44. The method of claim 39, wherein the bijel-templated material is produced by at least (i) forming a bijel from a mixture comprising a plurality of particles, a first liquid, and a second liquid, being different from the first liquid and including a monomer, which may be added after formation of the bijel, where the second liquid is partially miscible with the first liquid, and (ii) polymerizing the monomer-containing second liquid.

45. The method of claim 39, further comprising removing the plurality of particles and the first liquid after polymerization of the prepolymer mixture.

46. The method of claim 39, wherein the bijel-templated material comprises continuous, interconnected channels with multiple perfusion outlets.

47. A method of fabricating a biomedical device comprising:
a. placing a prepolymer mixture in a cannula; and
b. polymerizing the prepolymer mixture to form a bijel-templated material such that a portion of the bijel-templated material is bound to an internal surface of the cannula and a remaining portion of the bijel-templated material is protruding from an opening of the cannula.

48. The method of claim 47, wherein the bijel-templated material is produced by (i) forming a bijel from a mixture comprising a plurality of particles, a first liquid, and a second liquid, being different from the first liquid and including a monomer, which may be added after formation of the bijel, where the second liquid is partially miscible with the first liquid, and (ii) polymerizing the monomer-containing second liquid.

49. The method of claim 47, further comprising removing the plurality of particles and the first liquid after polymerization of the prepolymer mixture.

50. The method of claim 47, wherein the bijel-templated material comprises continuous, interconnected channels with multiple perfusion outlets.
